(19) United States
(12) Patent Application Publication
(10) Pub. No.: US 2024/0185947 A1
(43) Pub. Date: Jun. 6, 2024

(54) ARTIFICIAL KETOREDUCTASE VARIANTS AND THE DESIGN METHODOLOGY THEREOF

(71) Applicant: ENZYMASTER (NINGBO) BIO-ENGINEERING CO., LTD., Ningbo, Zhejiang (CN)

(72) Inventors: Shumin SHI, Ningbo, Zhejiang (CN); Junpeng XU, Ningbo, Zhejiang (CN); Marco BOCOLA, Ningbo, Zhejiang (CN); Baoqin CAI, Ningbo, Zhejiang (CN); Haibin CHEN, Ningbo, Zhejiang (CN); Mengying YU, Ningbo, Zhejiang (CN); Xiao LUO, Ningbo, Zhejiang (CN); Yafang LIANG, Ningbo, Zhejiang (CN); Kuifang HE, Ningbo, Zhejiang (CN); Qinli PENG, Ningbo, Zhejiang (CN)

(73) Assignee: ENZYMASTER (NINGBO) BIO-ENGINEERING CO., LTD., Ningbo, Zhejiang (CN)

(21) Appl. No.: 18/553,355

(22) PCT Filed: Apr. 20, 2022
(86) PCT No.: PCT/CN2022/087783

§ 371 Date: Sep. 29, 2023

(30) Foreign Application Priority Data
May 3, 2021 (CN) 202110486993.8
May 3, 2021 (CN) 202110487012.1

(51) Int. Cl. G16B 15/20; C12N 9/04; G16B 45/00

(52) U.S. Cl. CPC G16B 15/20; C12N 9/0006; G16B 45/00

(57) ABSTRACT

The present invention provides ketoreductase variants having activity on both (R)-(−)-1,3-butanediol and (S)-(+)-1,3-butanediol, and the computational methodology for designing the above mentioned ketoreductase variants. The ketoreductase variants disclosed in the present application can catalyze the reaction of synthesizing 4-hydroxy-2-butanone from racemic 1,3-butanediol, which has industrial application prospects.

### PRIORITY

This application corresponds to the U.S. National phase of International Application No. PCT/CN2022/087783, filed Apr. 20, 2022, which, in turn, claims priority to Chinese Patent Application No. 202110487012.1 filed May 3, 2021, and Chinese Patent Application No. 202110286993.8 filed May 3, 2021, the contents of which are incorporated by reference herein in their entirety.

### SEQUENCE LISTING

The instant application contains a Sequence Listing that has been submitted in ASCII format via EFS-Web and is hereby incorporated by reference in its entirety. Said ASCII copy, created on Sep. 29, 2023, is named LNK_249US_SL.txt and is 36,003 bytes in size.

### Technical Field of the Present Invention

The present invention relates to computer-aided design and virtual screening of protein variants, more specifically, it relates to a computational methodology that combines virtual screening for variant's stability and virtual screening for variant's catalytic activity to enable the design of enzyme variants with activity on non-natural substrates.

### Background of the Present Invention

Enzymes, a kind of catalyst in the form of protein, play an important role in the modern compound-synthesis industry. With the continuous expansion of enzymes' application, the catalytic performance of wild-type enzymes existing in nature can no longer meet the requirements of research and industrial application. Directed evolution is one of the most important technical methods for people to engineer enzymes. It is a rapid protein engineering strategy with certain target that works similarly as natural evolution, also known as laboratory evolution. Without knowledge of protein structure and reaction mechanism, an evolutionary process that would take millions of years in nature to obtain enzymes with desired functions can be accomplished in a relatively short time in the laboratory. In recent years, directed evolution has been widely used in the development of desired enzymes in the fields of pharmaceuticals, food, and chemical industry, which has triggered another revolution in the field of biocatalysis technology and greatly expanded the research and application scope of protein engineering. The applicant of this invention has been devoted to the research and application of the directed enzyme evolution and has successfully developed lots of enzyme catalysts for the synthesis of medicines and fine chemicals. However, directed enzyme evolution in laboratory usually requires the screening of a large number of enzyme variants, and the construction of these variants and subsequent screening work are burdensome tasks for researchers. On the basis of our previous studies on a large number of samples from directed evolution projects, coupled with computational biology and bioinformatics technology, the applicant developed a computational methodology that enables efficient virtual screening of envisaged enzyme variants and reliable prediction of enzyme variants with desired properties, thereby greatly reducing the size of enzyme mutant libraries that need to be constructed and screened in laboratory. The computational methodology disclosed in this invention is a powerful supplement to experimental work in wet lab for directed enzyme evolution; moreover, it can break the limitation of the size of enzyme mutant libraries that can be constructed and screened in the laboratory, which can reduce R&D costs, improve R&D efficiency, and more effectively obtain enzyme variants with desired properties.

Several computational methods have now been published for protein study. For example, there are molecular docking algorithms for observing the binding mode of small molecular substrates with proteins, homology modeling and de novo modeling algorithms for predicting protein 3D structures, and tools for calculating the structural stability of proteins. FoldX, I-mutant, Rosetta, etc., are methods that have been widely used in protein design. However, all these algorithms are kind of empirical mathematical calculations, in which the calculation formulas contain energy calculation terms of some physical principles as well as statistical energy terms obtained from existing databases. So far, the computer-aided methods for designing protein variants have various limitations, and the performance of the variants designed or predicted by these algorithms is hardly in agreement with the experimental results. Currently, there is no computational protocol to reliably design or predict enzyme variants with desired catalytic properties.

### SUMMARY OF THE PRESENT INVENTION

In particular, the present invention developed a computational method for efficient virtual screening of enzyme variants and reliable prediction of enzyme variants with desired properties. The present invention constructed a special method for processing the stability evaluation result, and creatively combined a process of calculating free energy barrier (using QM/MM method), which can improve the accuracy of the virtual screening of enzyme variants. The computational method disclosed by this invention may greatly reduce the number of variants to be constructed and tested in the wet lab, and thus save manpower and material resources. Unexpectedly, this method achieved the effect of enzyme engineering that cannot be achieved by traditional directed enzyme evolution methods.

### BRIEF DESCRIPTION OF THE DRAWINGS

FIG. 1 shows the workflow for virtual screening of enzyme variants.

FIG. 2 shows the schematic drawing to show Free Energy Barrie.

FIG. 3 shows conversion of racemic 1,3-butanediol to 4-hydroxy-2-butanone catalyzed by KRED.

FIG. 4 shows cadee process for Free Energy Barrier Calculation.

FIG. 5 shows the atom Number of (S)-(+)-1,3-butanediol.

FIG. 6 shows the conversion of 4-hydroxy-2-butanone to 1,3-butanediol catalyzed by KRED, with simultaneous conversion of isopropanol to acetone catalyzed by the same KRED to achieve regeneration of NADH.

FIG. 7 shows that the catalytic performance of the 10 KRED variants were tested using the experimental processes as follow.

FIG. 8 shows the GC spectra of 4-hydroxy-2-butanone and 1,3-butanediol.

FIG. 9 shows the GC spectra of (R)-(−)-1,3-butanediol and (S)-(−)-1,3-butanediol.

### DETAILED DESCRIPTION OF THE PREFERRED EMBODIMENTS

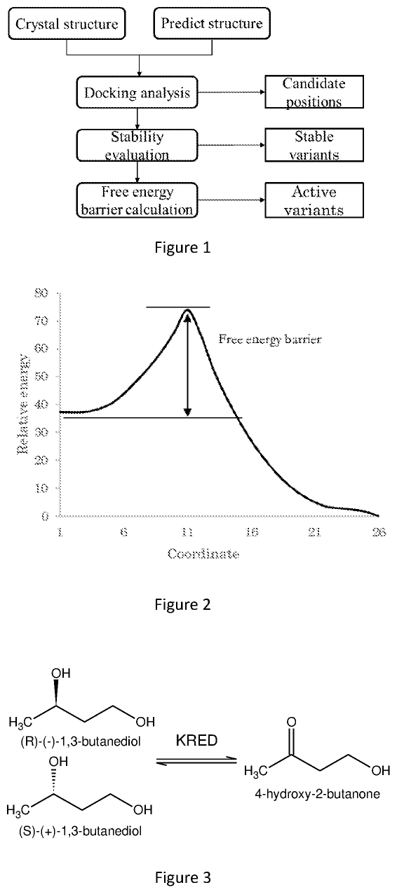

The computational method of the present invention, as shown in FIG. 1, includes the following four specific steps:

(1) Obtaining the 3D protein structure of the target enzyme. The 3D protein structure could be a structure obtained from experimental studies that have been recorded in the PDB (Protein Data Bank) database, or it could be a structure predicted by homology modeling or de novo modeling methods. Homology modeling method using highly homologous template structure would be more accurate than de novo modeling methods. 3D Protein structures obtained from different sources should be in a catalytic conformation, that is, the protein-ligand (substrate, product or transition state) complex.

(2) Docking analysis. Determine the sites of the target enzyme structure that bind to the ligand, and then dock the enzyme's natural substrate and/or target substrates (including non-natural substrates) with the protein. According to the different binding conformations of the natural substrate and/or the target substrates, the appropriate positions in amino acid sequence of the target enzyme are selected as candidate positions for mutagenesis (i.e. amino acid substitutions). Currently, there are many software that can perform molecular docking, such as Rosetta, Discovery studio, Schrodinger, Yasara (including Autodock and Autodock vina plugins) and so on. The amino acid positions that need to be mutated were screened by aligning the docking conformations.

(3) Stability evaluation. Stability of enzyme variants with amino acid substitutions on candidate positions from the step (2) will be evaluated with algorithms. First, a python script is used to produce (in batch mode) a collection of enzyme variants for virtual screening, and the 3D structure of each variant will be then generated using software such as Yasara or Rosetta; then, the stability of each variant will be evaluated with algorithms such as ddg_monomer, Cartesian_ddg, FoldX, Provean, ELASPIC or Amber TI; finally, the free energy difference (ΔΔG) between the structure of each variant and that of the starting enzyme is calculated using a python analysis script.

There are two strategies to process the ΔΔG results: a simple sorting method [3a] and a statistical method [3b].

[3a] Simple sorting method: That is, the ΔΔG results of all variants are simply sorted from low to high in terms of numeric values, and the top-ranked variants are selected as the stable variants obtained by computer virtual screening.

[3b] Statistical method: First, the ΔΔG results of all variants are sorted from low to high in terms of numeric values; second, amino acid substitutions of some top-ranked variants (i.e. stable cluster) and amino acid substitutions of some bottom-ranked variants (i.e. unstable cluster) are selected for frequency analysis. For a specific amino acid position, the substitution with higher frequency in unstable cluster is subtracted from the substitutions with higher frequency in stable cluster to obtain the theoretically stable substitutions at this position (i.e. predicted beneficial substitutions). Finally, the stable substitutions at each position obtained in such way will be combined to give stable variants as predicted by computer virtual screening.

Judgment criteria of stability evaluation results: ΔΔG≤−1 kcal/mol is regarded as a stable variant; ΔΔG≥1 kcal/mol is regarded as an unstable variant; −1 kcal/mol<ΔΔG<1 kcal/mol is regarded as a stability-neutral variant.

There is many software to run algorithms for evaluating the stability of 3D protein structure, so it is not limited to the Yasara, Rosetta or Discovery studio in this step. The creative contribution of the present invention is the conception and adoption of the statistical method [3b]. Statistical analysis is used to identify beneficial substitutions in terms of structural stability at each amino acid position, and then these substitutions are combined to generate multiple-site-substitution stable variants predicted by computer virtual screening.

Most virtual screening methods stop at step (3), i.e. obtaining the stable variants predicted by virtual screening. These stable variants will be then tested and verified in the laboratory with specific experimental protocols, and no further computational methods will be used to assess the catalytic activity of these candidates. In addition, due to the limitation of stability evaluation, the stable variants sorted by the simple sorting method [3a] would miss some important multiple-site-substitution variants. The present invention provides a statistical method [3b] to screen for stable variants, including variants that otherwise cannot be predicted by other virtual screening methods. More importantly, virtual screening methods currently available in the art cannot meet the desire to further determine the catalytic activity of predicted stable variants, let alone evaluate whether predicted stable variants are active on non-natural substrates.

The computational methodology disclosed in the present invention further includes the method and process for calculating free energy barrier of each variant in catalyzing specific chemical reactions, which realizes the prediction of the catalytic activity of the stable variant from step (3). Therefore, it enables to evaluate whether the predicted stable variants have activity on non-natural substrates.

(4) Free energy barrier calculation. This approach is based on the force field description of the reaction states for different substrates in combination with the quantum mechanical description of chemical reactions within the framework of valence bond theory. It allows free energy barrier calculations to take advantage of the fast speed of classical force-field-based methods, and at the same time to carry a lot of chemical and thermodynamic information, resulting in meaningful physical descriptions of bond formation and bond breaking processes. The preparatory work of the free energy barrier calculation includes selecting the force field, determining the rate-limiting step of the target reaction, and the transition state, etc. After the calculation parameters are determined, the free energy barrier calculation is implemented in a “cadee” process, and Qtools is used to analyze the calculation results in the present invention. In the cadee process, for example, a default setting is 12.6 ns of the simulation calculation time, the systems would be heated gradually from 0.01 to 300 K over the course of 90 ps of simulation time, starting with a 200 kcal·mol⁻¹·Å⁻² harmonic restraint on all protein atoms and 20 kcal·mol⁻¹·Å⁻² on all water atoms in the simulation sphere. As the temperature increases, the harmonic restraint is gradually decreased. The temperature is regulated using the Berendsen thermostat. A 1 fs time step is used, and the reaction coordinate is set to λ=0.5 for all simulations to start the subsequent free energy barrier calculations of the reaction step close to the transition state. Molecular dynamics (MD) simulations at 8 ns is performed for each replica in the four parallel calculations, and the simulation results are used as the starting point for empirical valence bond simulations. Snapshots at every 1 ns of the 8 ns long MD simulation are taken to get structures close to the transition state which are used to run an empirical valence bond simulation of 520 ps in length, distributed over 26 EVB-FEP/US windows of 20 ps each (λ=0, 0.05, 0.075, 0.1, 0.125, 0.15, 0.2, 0.25, 0.30, 0.35, 0.40, 0.425, 0.45, 0.55, 0.575, 0.6, 0.65, 0.70, 0.75, 0.80, 0.85, 0.875, 0.90, 0.925, 0.95, 1). Additionally, for the data mapping of each snapshot, we would use the data of the preceding 1 ns MD simulation (where λ=0.5) to achieve an increased sampling close to the transition state. If the default setting of cadee's workflow is used, it could be a very long calculation process. Therefore, the MD simulation time in the calculation process of the present invention is reduced from 8 ns to 4 ns and the repeated calculation cycle in each parallel calculation replica is reduced to 4. In this way, the calculation time can be reduced to less than 24 hours, and the calculation accuracy does not change much. This multi-task parallel computing in our cadee process can be performed on a multi-core computer, so the virtual screening of catalytic activity for a large number of variants can be achieved using a high-performance computer.

The free energy barrier is the minimum energy required by the reacting molecule to reach the activated state, and the magnitude of the energy barrier can reflect the difficulty of the reaction. As shown in FIG. 2, the difference between the potential energy of the enzyme & the substrate in the free state and the potential energy of the enzyme-substrate complex in activated state is the free energy barrier; in other words, it is the gap from the lowest energy point (the optimal conformation of enzyme & substrate in free state) to the highest energy point (the optimal conformation of the enzyme-substrate complex in activated state). In our calculation process, it is the difference between the lowest energy of the initial state and the highest energy of the activated state.

Following the virtual screening workflow as shown in FIG. 1 with the abovementioned methods, a small set of enzyme variants are predicted and subsequently subject to experimental verification. This small set of predicted enzyme variants are constructed and expressed in the laboratory, and their catalytic activity on the target substrates are tested to check whether they have the expected catalytic activities.

In the usual practice of directed enzyme evolution, the study of 3D protein structure and simulation of enzymatic reaction mechanism is not performed for the target enzyme, so it is hardly to predict which amino acid substitutions at which positions are beneficial to improve the enzyme's performance. In order to screening for beneficial amino acid substitutions (or mutations), it is often necessary to building large and diverse libraries, and the screening of these libraries take a lot of time and laboratory resources. When the computational methodology in the present invention is implemented, the stability of the enzyme variants can be evaluated by means of virtual screening based on the docking structure of the enzyme and the substrate in combination with the result processing method disclosed herein. A large number of unstable variants can be filtered out with this virtual screening, so the number of variants for laboratory screening can be greatly reduced. Regarding the calculation for enzyme stability, the calculation itself uses empirical algorithms and does not guarantee complete accuracy; for the feasibility of the calculation, many factors such as the elastic change of the amino acid backbone are not regarded as variables, small molecule ligands in substrate docking step are often docked as rigid bodies, and the surrounding environmental factors are also simplified. In addition, in the currently known virtual screening methods, the stability calculation results are processed by directly sorting the energy values to give beneficial mutations (with the lowest energy). Due to these limitations, some beneficial mutations could be missed by virtual screening methods. In order to overcome these limitations, the applicant, based on years of experience and data in directed enzyme evolution, has been improving the stability virtual screening and the method to process calculation results. Especially, a statistical method to process ΔΔG results of virtually screening variants is proposed in the present invention, which allows to come up with beneficial mutations or combination of beneficial mutations that are often ignored in other virtual screening algorithms.

According to the energy function parameters of calculation algorithms, the most computational protocols for protein design are only to evaluate the structural stability of given variants, and rarely involve the activity assessment of enzyme variants for catalyzing a specific reaction. The stability evaluation of the overall structure of enzyme variants is one aspect in enzyme engineering and is a prerequisite for enzymes' catalytic activity. The discovery, creation and improvement of the catalytic activity of enzymes is actually the main focus in the biocatalysis industry, simply because the availability of highly active enzymes for given substrates enables industrial application of biocatalysis. However, most computational protocols fail to achieve the assessment of the catalytic activity for given substrates after the stability evaluation. Therefore, on top of optimizing the stability evaluation method, the present invention further developed and incorporated a free energy barrier calculation to assess the activity of given enzyme variants for catalyzing a specific reaction (especially the activities for non-natural substrates). By taking multiple factors into consideration, such as substrate, intermediate and transition state, etc., the computational methodology disclosed in the present invention allows the assessment of catalytic activity of predicted stable variants. The virtual screening method disclosed in the present invention can be used to design high-quality, small-sized libraries of enzyme variants for experimental screening in wet lab, which greatly improves the accuracy and efficiency of enzyme virtual screening; the activity evaluation of predicted stable variants using free energy barrier calculation can greatly improve the efficiency and effectiveness of enzyme engineering.

### EXAMPLES

The following example further illustrate the present invention, giving a clear description of the technical schemes of the present invention. The present invention is not limited thereto. The following example are merely part of the embodiments of the present invention, not all of them. Based on the example in the present invention, all other examples obtained by ordinary technical personnel in this field without making creative effort fall within the scope of the present invention.

### Example: Design of a Ketone Reductase (KRED) Variant that is Active to a Non-Natural Substrate (the Opposite Enantiomer to its Natural Substrate)

The ketone reductase disclosed in patent CN111321129A, can asymmetrically convert 4-hydroxy-2-butanone to (R)-(−)-1,3-butanediol. Meanwhile, this enzyme can also catalyze the reverse reaction to convert alcohol to ketone; due to the strict specificity of this enzyme, it is only active to (R)-(−)-1,3-butanediol but is not active to its opposite enantiomer, i.e. (S)-(+)-1,3-butanediol. If racemic 1,3-butanediol is used as the substrate (mixture of (R)-(−)-1,3-butanediol and (S)-(+)-1,3-butanediol at 1:1 ratio), (R)-(−)-1,3-butanediol is converted to 4-hydroxy-2-butanone by this enzyme, while (S)-(+)-1,3-butanediol remains intact, resulting in a low overall yield of 4-hydroxy-2-butanone from racemic 1,3-butanediol. The development of a keto reductase variant that can convert all racemic 1,3-butanediol to 4-hydroxy-2-butanone would be of great industrial application. In order to develop such a variant, the ketone reductase disclosed in CN111321129A was used as a starting template, its amino acid sequence is shown as SEQ ID No: 2, and its DNA sequence is shown as SEQ ID No: 1. SEQ ID No: 2 has a stereoselectivity of >99% (in terms of enantiomer excess) to (R)-(−)-1,3-butanediol but has no activity towards (S)-(+)-1,3-butanediol; that is, (S)-(+)-1,3-butanediol is an non-natural substrate for SEQ ID No: 2.

Using the computational protocols disclosed in the present invention, ten variants were predicted to exhibit activity towards both (R)-(−)-1,3-butanediol and (S)-(+)-1,3-butanediol. These ten predicted variants were tested by experimental assays in wet lab, variants that retain high catalytic activity and can convert both (R)-(−)-1,3-butanediol and (S)-(+)-1,3-butanediol into 4-hydroxy-2-butanone simultaneously were identified, as shown in FIG. 3.

The computational design of KRED variants for converting racemic 1,3-butanediol to 4-hydroxy-2-butanone.

(1) Obtaining the protein structure: Homology modeling of SEQ ID NO: 2 with YASARA software to obtain the 3D structural model of the target protein.

(2) Docking analysis: (R)-(−)-1,3-butanediol and (S)-(+)-1,3-butanediol were docked with the target protein respectively using Yasara software. And then the results were analyzed. The docking results of (S)-(+)-1,3-butanediol showed excessive steric hindrance of the residues' side chain of amino acids at the positions 1144, H145, Q150, Y188 etc. Therefore, in this example, these positions were selected as mutagenesis sites for virtual screening.

(3) Stability evaluation: The candidate substitutions were selected for each position of 1144, H145, Q150, Y188, as shown in Table 1. Then a python script was used to generate the input file of mutation combinations required by Rosetta, and there were 400 variants for possible mutation combinations. The free energy difference (ΔΔG) between each variant structure and that of SEQ ID NO: 2's structure was calculated using the Cartesian_ddg algorithm.

TABLE 1

| Position | Substitution |
|---|---|
| I144 | ASLG |
| H145 | ACG |
| Q150 | NAS |
| Y188 | AGST |

Afterwards, the statistical method [3b] was used to process the calculation results, and the predicted beneficial substitutions are shown in Table 2.

TABLE 2

| Positions | X144 | X145 | X150 | X188 |
|---|---|---|---|---|
| Predicted beneficial | I | H | Q | G |
| substitutions | G | C | N | A |
| | A | — | — | Y |

(4) Free energy barrier calculation: Combining the predicted beneficial substitutions in step (3) gives 36 possible variants in total, and cadee process was used to calculate the free energy barrier.

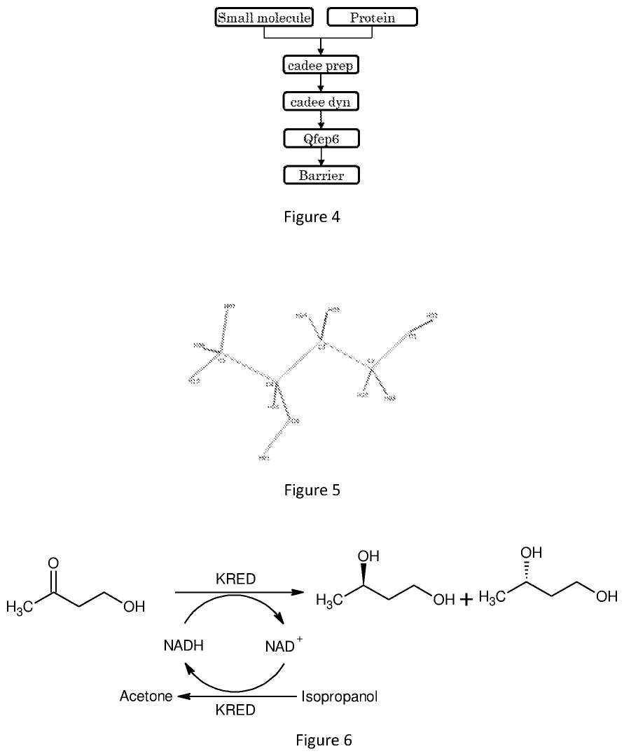

Simulation settings: The simulation system was first dissolved in spherical water droplets of TIP3P model water molecules with a radius of 20 Å centered on the C3 atom of (S)-(+)-1,3-butanediol, where all atoms could move freely within 17 Å from the simulation center (i.e. the C3 atom of (S)-(+)-1,3-butanediol). All atoms located between 17 Å and 20 Å from the simulation center were constrained with a 10 kcal·mol⁻¹·Å⁻² harmonic-restraint, and atoms beyond 20 Å were constrained with a harmonic force constant of 200 kcal. H atoms were confined in the solvent using the SHAKE algorithm. A cutoff of 10 Å was used to calculate non-bonding interactions between all atoms except those in the empirical valence region, all of which were explicitly calculated to be 99 Å. All long-range electrostatics above this critical value were treated using the Local Reaction Field (LRF) method, cadee process for Free Energy Barrier Calculation is shown in FIG. 4, atom number of (S)-(+)-1,3-butanediol is shown in FIG. 5.

10 variants with the lowest barrier were selected based on the calculation results from cadee process, and they are shown in Table 3.

TABLE 3

| Amino acid sequence # of KRED | Mutations compared to |
|---|---|
| variants | SEQ ID NO: 2 |
| SEQ ID NO: 4 | I144G |
| SEQ ID NO: 6 | Y188A |
| SEQ ID NO: 8 | Y188G |
| SEQ ID NO: 10 | H145C |
| SEQ ID NO: 12 | H145C; Y188A |
| SEQ ID NO: 14 | H145C; Y188G |
| SEQ ID NO: 16 | I144G; H145C |
| SEQ ID NO: 18 | I144G; H145C; Y188A |
| SEQ ID NO: 20 | I144G; H145C; Y188G |
| SEQ ID NO: 22 | I144G; Y188A |

### (5) Experimental Validation

(5.1) The stereoselectivity towards 1,3-butanediol of the 10 variants shown in Table 3 were studied using the reaction shown in FIG. 6.

To a reaction flask, 0.1 g 4-hydroxy-2-butanone, 0.5 mL isopropanol, 0.1 g wet cells expressing each KRED variant (refer to the method disclosed in patent CN111321129 Å for recombinant expression process), and 0.005 g NAD⁺ were added. The final reaction volume in the reaction flask was filled up to 5 mL by 0.1 M PB (pH7) buffer. The reaction flask was placed on an IKA magnetic stirrer at 40° C., and the stirring speed was set to 400 rpm to start the reaction. After the reaction was carried out for 1 hour, the reaction was sampled and measured by GC. The ee % (enantiomeric excess) values of produced 1,3-butanediol are shown in Table 4. The calculation formula is ee %=([R]−[S])/([R]+[S]), [R] represents the concentration of (R)-(−)-1,3-butanediol in the sample, and [S] represents the concentration of (S)-(+)-1,3-butanediol in the sample.

TABLE 4

| Amino acid | | |
|---|---|---|
| sequence # of KRED | | |
| variants | Corresponding DNA sequence # | ee % |
| SEQ ID NO: 2 | SEQ ID NO: 1 | >99.9%* |
| SEQ ID NO: 4 | SEQ ID NO: 3 | 30.2% |
| SEQ ID NO: 6 | SEQ ID NO: 5 | 12.7% |
| SEQ ID NO: 8 | SEQ ID NO: 7 | 4.6% |
| SEQ ID NO: 10 | SEQ ID NO: 9 | 36.8% |
| SEQ ID NO: 12 | SEQ ID NO: 11 | −4.9% |
| SEQ ID NO: 14 | SEQ ID NO: 13 | 0.6% |
| SEQ ID NO: 16 | SEQ ID NO: 15 | 8.5% |
| SEQ ID NO: 18 | SEQ ID NO: 17 | 36.1% |
| SEQ ID NO: 20 | SEQ ID NO: 29 | 17.7% |
| SEQ ID NO: 22 | SEQ ID NO: 21 | 56.9% |

*Note:
(S)-(+)-1,3-butanediol was not detected.

The ee % results in Table 4 show that both (R)-(−)-1,3-butanediol and (S)-(+)-1,3-butanediol were produced by the 10 KRED variants (i.e. SEQ ID NO: 4, 6, 8, 10, 12, 14, 16, 18, 20, 22), suggesting that the computationally predicted 10 KRED variants possess the activity towards (S)-(+)-1,3-butanediol.

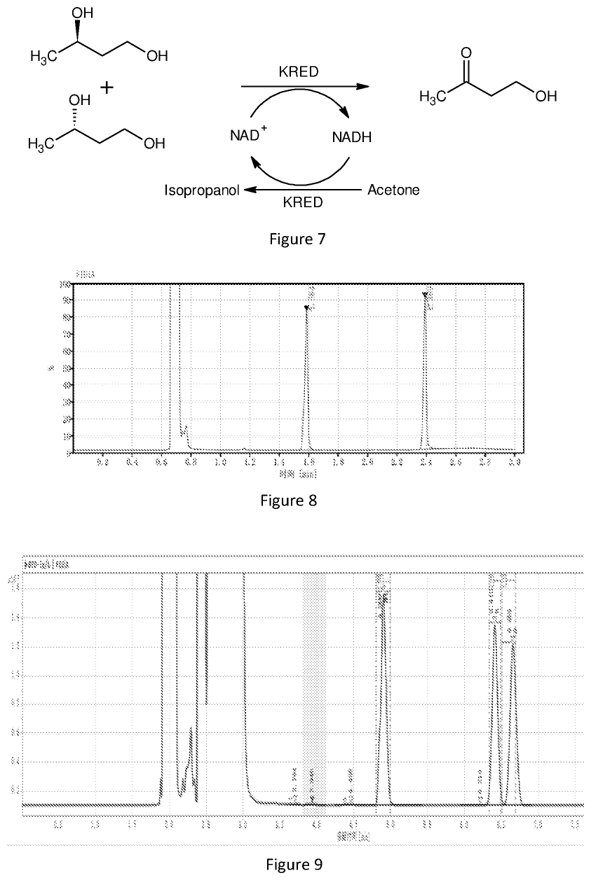

(5.2) For converting the racemic 1,3-butanediol to 4-hydroxy-2-butanone as shown in FIG. 7, the catalytic performance of the 10 KRED variants were tested using the experimental processes as follows.

To a reaction flask, 0.1 g racemic 1,3-butanediol ((R)-(−)-1,3-butanediol and (S)-(+)-1,3-butanediol mixture of 1:1 ratio), 0.5 mL acetone, 0.1 g wet cells expressing each KRED variant (refer to the method disclosed in patent CN111321129 Å for recombinant expression process), and 0.005 g NAD⁺ cofactor were added. The final reaction volume in the reaction flask was filled up to 5 mL by 0.1 M PB (pH7) buffer.

The reaction flask was placed on an IKA magnetic stirrer at 40° C., and the stirring speed was set to 400 rpm to start the reaction. After the reaction was carried out for 1 hour, the reaction was sampled and measured by GC. The conversion of 1,3-butanediol is shown in Table 5.

TABLE 5

| | Conversion of racemic 1,3- |
|---|---|
| KRED variants | butanediol |
| SEQ ID NO: 2 | 45.1% |
| SEQ ID NO: 4 | 61.1% |
| SEQ ID NO: 6 | 87.5% |
| SEQ ID NO: 8 | 89.2% |
| SEQ ID NO: 10 | 55.8% |
| SEQ ID NO: 12 | 84.7% |
| SEQ ID NO: 14 | 80.9% |
| SEQ ID NO: 16 | 81.5% |
| SEQ ID NO: 18 | 60.7% |
| SEQ ID NO: 20 | 77.9% |
| SEQ ID NO: 22 | 51.1% |

As SEQ ID NO: 2 only shows activity to (R)-(−)-1,3-butanediol, and no activity to (S)-(+)-1,3-butanediol, the theoretical maximum conversion that SEQ ID NO: 2 can achieve for the reaction shown in FIG. 7 is 50%. The data in Table 5 showed that the computationally designed 10 KRED variants (SEQ ID NO: 4, 6, 8, 10, 12, 14, 16, 18, 20, 22) can reach >50% conversion, which suggests that these variants can convert both (R)-(−)-1,3-butanediol and (S)-(+)-1,3-butanediol to 4-hydroxy-2-butanone.

### Analytical Methods:

GC method for conversion analysis: The chromatographic column was DB-WAX 15m*0.25 mm*0.25 μm, the carrier gas was N₂, the detector was FID, the inlet temperature was 250° C., the split ratio was 28:1, and the detector temperature was 300° C. The injection volume was 1 μL, the column temperature was 130° C., the temperature was raised to 150° C. at 10° C./min and then raised to 160° C. at 20° C./min, wherein the retention time of 4-hydroxy-2-butanone was 1.5 min, and the retention time of 1,3-butanediol was 2.3 min, as shown in FIG. 8.

GC method for chiral analysis: Before injection, sample was derivatized as following: 50 μL MSTFA and 30 μL anhydrous pyridine were added to 200 μL of quenched sample and mixed well in a 1.5 mL centrifuge tube, and the derivatization reaction was shaken for 30 min. The chromatographic column was CP-Chirasil Dex CB (CP7502) 25m*0.25 mm*0.25 μm, the carrier gas was N₂, the detector was FID, the inlet temperature was 250° C., the split ratio was 28:1, and the detector temperature was 300° C. The injection volume was 1 μL, the column temperature was 105° C., and stop time was 9 minutes, wherein the retention time of (R)-(−)-1,3-butanediol was 6.4 min, and the retention time of (S)-(+)-1,3-butanediol was 6.6 min, as shown in FIG. 9.

It should be understood that after reading the above contents of the present invention, those skilled in the art may make various modifications or changes to the present invention. And these equivalent forms also fall within the scope of the appended claims of the present invention.

1-9. (canceled)

10. A method, implemented using a computer system comprising one or more processors and system memory, for designing artificial variants of a given enzyme having catalytic activity for non-natural substrate(s), said method comprising the steps of:
(1) introducing into said computer system a structural model for said given enzyme, wherein said structural model comprises a three dimensional computational representation of said enzyme in a catalytic conformation;
(2) using said one or more processors to computationally model enzyme-substrate binding, determine substrate binding site(s) and binding conformation(s) for said given enzyme, and generate a three-dimensional computational representation of an enzyme-substrate complex;
(3) using said one or more processors to perform systematic molecular docking analyses on the enzyme-substrate complex generated in step (2) to identify candidate positions for enzyme mutagenesis specifying amino acid substitutions;
(4) using said one or more processors to virtually screen on the basis of protein structure stability evaluation methods all possible combinations of specified amino acid substitutions of all candidate positions to predict beneficial substitutions for each candidate position; and
(5) using said one or more processors to virtually screen on the basis of free energy barrier calculations all possible combinations of predicted beneficial substitutions of all candidate positions identified in step (4) to identify catalytically active variants for a given enzyme.

11. The method according to claim 1, wherein results of the evaluation methods for protein structure stability utilized in step (4) are processed by a statistical method comprising the following steps:
(i) determine the free energy difference (ΔΔG) of all calculated variants and sort from low to high in terms of numeric values, wherein a high numeric value corresponds to a stable cluster and a low numeric value corresponds to an unstable cluster;
(ii) select a number of top-ranked stable clusters and bottom-ranked unstable clusters for frequency analysis wherein, for a specific amino acid position, an amino acid substitution with a higher frequency in an unstable cluster is subtracted from amino acid substitutions having a higher frequency in stable cluster to obtain the theoretically stable substitutions at the specific position; and
(iii) combine the substitutions at each position determined to be stable in step (ii) to obtain a set of stable variants that correspond to predicted beneficial substitutions as predicted by computer virtual screening.

12. An artificial variant designed using the method of claim 1, wherein said artificial variant is a ketoreductase variant and is active to both (R)-(−)-1,3-butanediol and (S)-(+)-1,3-butanediol.

13. The method according to claim 1, wherein the three-dimensional computational representation of an enzyme-substrate complex of step (2) is generated using software selected from the group consisting of Yasara, Discovery studio and Rosetta.

14. The method according to claim 1, wherein the evaluation methods for protein structure stability utilized in step (4) are selected from the group consisting of ddg_monomer, Cartesian_ddg, FoldX, Provean, ELASPIC and Amber TI.

15. The method according to claim 1, wherein the “free energy barrier” is defined as the energy difference between the lowest energy point, which corresponds to the optimal conformation of an enzyme and substrate in a free state, and the highest energy point, which corresponds to the optimal conformation an enzyme-substrate complex in an activated state.

16. A ketoreductase variant obtained by the method of claim 1, wherein said ketoreductase variant catalyzes the synthesis of 4-hydroxy-2-butanone from (R)-(−)-1,3-butanediol and (S)-(+)-1,3-butanediol simultaneously under appropriate reaction conditions, further wherein the amino acid sequence of said variant contains the amino acid substitutions X145C, X188G or X188 Å as compared to the sequence of SEQ ID NO: 2.

17. The ketoreductase variant according to claim 7, wherein the amino acid sequence of said ketoreductase variant further comprises the amino acid substitution X144G as compared to the sequence of SEQ ID NO: 2.

18. The ketoreductase variant according to claim 7, wherein the amino acid sequences of said ketoreductase variant is set forth in SEQ ID NO: 4, 6, 8, 10, 12, 14, 16, 18, 20, or 22.


SEQUENCE LISTING (22 sequences)

SEQ ID NO: 1 - DNA, 747 bp, Artificial Sequence
  Engineered sequence
atgggtatcctggacaacaaagtcgcactggttacgggcgctggttcgggcatcggtctggcggtggcacactcctacgctaaagaaggcgctaaagtcattgtgtcagatatcaacgaagacaagggtaataaaaccgttgaagatattaaagcacagggcggtgaagctagttttgtgaaagcggacaccagcaacccggaagaagtggaagccctggttaaacgtacggtcgaaatttatggtcgcctggatgtggcatgcaacaatgctggcattgcgggtgaaaaggcactggctggtgattacggcctggacagctggcgtaaagttctgtctgtgaatctggacggtgtcttctatggctgtaaatacgaactggaacaaatggagaaaaacggcggtggcgttatcgtcaatatggccagcattcatggtatcgttgcgcagccgctgaactctgcatatacctctgcgaaacacgccgtggttggcctgacgaaaaacattggtgctgattatggccagaaaaacatccgttgcaatgcggtgtgcccgggctacattgaaaccccgctgctggaatcactgacgaaagaaatgaaagaagccctgatctcgaaacacccgatgggtcgcctgggcaaaccggaagaagtggcagaactggttctgtttctgagttccgaaaaatcatcgttcatgaccggtggctattacctggtcgatggtggctacacggcagtg

SEQ ID NO: 2 - protein, 249 aa, Artificial Sequence
  Engineered sequence
MGILDNKVALVTGAGSGIGLAVAHSYAKEGAKVIVSDINEDKGNKTVEDIKAQGGEASFVKADTSNPEEVEALVKRTVEIYGRLDVACNNAGIAGEKALAGDYGLDSWRKVLSVNLDGVFYGCKYELEQMEKNGGGVIVNMASIHGIVAQPLNSAYTSAKHAVVGLTKNIGADYGQKNIRCNAVCPGYIETPLLESLTKEMKEALISKHPMGRLGKPEEVAELVLFLSSEKSSFMTGGYYLVDGGYTAV

SEQ ID NO: 3 - DNA, 747 bp, Artificial Sequence
  Engineered sequence
atgggtatcctggacaacaaagtcgcactggttacgggcgctggttcgggcatcggtctggcggtggcacactcctacgctaaagaaggcgctaaagtcattgtgtcagatatcaacgaagacaagggtaataaaaccgttgaagatattaaagcacagggcggtgaagctagttttgtgaaagcggacaccagcaacccggaagaagtggaagccctggttaaacgtacggtcgaaatttatggtcgcctggatgtggcatgcaacaatgctggcattgcgggtgaaaaggcactggctggtgattacggcctggacagctggcgtaaagttctgtctgtgaatctggacggtgtcttctatggctgtaaatacgaactggaacaaatggagaaaaacggcggtggcgttatcgtcaatatggccagcggccatggtatcgttgcgcagccgctgaactctgcatatacctctgcgaaacacgccgtggttggcctgacgaaaaacattggtgctgattatggccagaaaaacatccgttgcaatgcggtgtgcccgggctacattgaaaccccgctgctggaatcactgacgaaagaaatgaaagaagccctgatctcgaaacacccgatgggtcgcctgggcaaaccggaagaagtggcagaactggttctgtttctgagttccgaaaaatcatcgttcatgaccggtggctattacctggtcgatggtggctacacggcagtg

SEQ ID NO: 4 - protein, 249 aa, Artificial Sequence
  Engineered sequence
MGILDNKVALVTGAGSGIGLAVAHSYAKEGAKVIVSDINEDKGNKTVEDIKAQGGEASFVKADTSNPEEVEALVKRTVEIYGRLDVACNNAGIAGEKALAGDYGLDSWRKVLSVNLDGVFYGCKYELEQMEKNGGGVIVNMASGHGIVAQPLNSAYTSAKHAVVGLTKNIGADYGQKNIRCNAVCPGYIETPLLESLTKEMKEALISKHPMGRLGKPEEVAELVLFLSSEKSSFMTGGYYLVDGGYTAV

SEQ ID NO: 5 - DNA, 747 bp, Artificial Sequence
  Engineered sequence
atgggtatcctggacaacaaagtcgcactggttacgggcgctggttcgggcatcggtctggcggtggcacactcctacgctaaagaaggcgctaaagtcattgtgtcagatatcaacgaagacaagggtaataaaaccgttgaagatattaaagcacagggcggtgaagctagttttgtgaaagcggacaccagcaacccggaagaagtggaagccctggttaaacgtacggtcgaaatttatggtcgcctggatgtggcatgcaacaatgctggcattgcgggtgaaaaggcactggctggtgattacggcctggacagctggcgtaaagttctgtctgtgaatctggacggtgtcttctatggctgtaaatacgaactggaacaaatggagaaaaacggcggtggcgttatcgtcaatatggccagcattcatggtatcgttgcgcagccgctgaactctgcatatacctctgcgaaacacgccgtggttggcctgacgaaaaacattggtgctgattatggccagaaaaacatccgttgcaatgcggtgtgcccgggcgcgattgaaaccccgctgctggaatcactgacgaaagaaatgaaagaagccctgatctcgaaacacccgatgggtcgcctgggcaaaccggaagaagtggcagaactggttctgtttctgagttccgaaaaatcatcgttcatgaccggtggctattacctggtcgatggtggctacacggcagtg

SEQ ID NO: 6 - protein, 249 aa, Artificial Sequence
  Engineered sequence
MGILDNKVALVTGAGSGIGLAVAHSYAKEGAKVIVSDINEDKGNKTVEDIKAQGGEASFVKADTSNPEEVEALVKRTVEIYGRLDVACNNAGIAGEKALAGDYGLDSWRKVLSVNLDGVFYGCKYELEQMEKNGGGVIVNMASIHGIVAQPLNSAYTSAKHAVVGLTKNIGADYGQKNIRCNAVCPGAIETPLLESLTKEMKEALISKHPMGRLGKPEEVAELVLFLSSEKSSFMTGGYYLVDGGYTAV

SEQ ID NO: 7 - DNA, 747 bp, Artificial Sequence
  Engineered sequence
atgggtatcctggacaacaaagtcgcactggttacgggcgctggttcgggcatcggtctggcggtggcacactcctacgctaaagaaggcgctaaagtcattgtgtcagatatcaacgaagacaagggtaataaaaccgttgaagatattaaagcacagggcggtgaagctagttttgtgaaagcggacaccagcaacccggaagaagtggaagccctggttaaacgtacggtcgaaatttatggtcgcctggatgtggcatgcaacaatgctggcattgcgggtgaaaaggcactggctggtgattacggcctggacagctggcgtaaagttctgtctgtgaatctggacggtgtcttctatggctgtaaatacgaactggaacaaatggagaaaaacggcggtggcgttatcgtcaatatggccagcattcatggtatcgttgcgcagccgctgaactctgcatatacctctgcgaaacacgccgtggttggcctgacgaaaaacattggtgctgattatggccagaaaaacatccgttgcaatgcggtgtgcccgggcggcattgaaaccccgctgctggaatcactgacgaaagaaatgaaagaagccctgatctcgaaacacccgatgggtcgcctgggcaaaccggaagaagtggcagaactggttctgtttctgagttccgaaaaatcatcgttcatgaccggtggctattacctggtcgatggtggctacacggcagtg

SEQ ID NO: 8 - protein, 249 aa, Artificial Sequence
  Engineered sequence
MGILDNKVALVTGAGSGIGLAVAHSYAKEGAKVIVSDINEDKGNKTVEDIKAQGGEASFVKADTSNPEEVEALVKRTVEIYGRLDVACNNAGIAGEKALAGDYGLDSWRKVLSVNLDGVFYGCKYELEQMEKNGGGVIVNMASIHGIVAQPLNSAYTSAKHAVVGLTKNIGADYGQKNIRCNAVCPGGIETPLLESLTKEMKEALISKHPMGRLGKPEEVAELVLFLSSEKSSFMTGGYYLVDGGYTAV

SEQ ID NO: 9 - DNA, 747 bp, Artificial Sequence
  Engineered sequence
atgggtatcctggacaacaaagtcgcactggttacgggcgctggttcgggcatcggtctggcggtggcacactcctacgctaaagaaggcgctaaagtcattgtgtcagatatcaacgaagacaagggtaataaaaccgttgaagatattaaagcacagggcggtgaagctagttttgtgaaagcggacaccagcaacccggaagaagtggaagccctggttaaacgtacggtcgaaatttatggtcgcctggatgtggcatgcaacaatgctggcattgcgggtgaaaaggcactggctggtgattacggcctggacagctggcgtaaagttctgtctgtgaatctggacggtgtcttctatggctgtaaatacgaactggaacaaatggagaaaaacggcggtggcgttatcgtcaatatggccagcatttgcggtatcgttgcgcagccgctgaactctgcatatacctctgcgaaacacgccgtggttggcctgacgaaaaacattggtgctgattatggccagaaaaacatccgttgcaatgcggtgtgcccgggctacattgaaaccccgctgctggaatcactgacgaaagaaatgaaagaagccctgatctcgaaacacccgatgggtcgcctgggcaaaccggaagaagtggcagaactggttctgtttctgagttccgaaaaatcatcgttcatgaccggtggctattacctggtcgatggtggctacacggcagtg

SEQ ID NO: 10 - protein, 249 aa, Artificial Sequence
  Engineered sequence
MGILDNKVALVTGAGSGIGLAVAHSYAKEGAKVIVSDINEDKGNKTVEDIKAQGGEASFVKADTSNPEEVEALVKRTVEIYGRLDVACNNAGIAGEKALAGDYGLDSWRKVLSVNLDGVFYGCKYELEQMEKNGGGVIVNMASICGIVAQPLNSAYTSAKHAVVGLTKNIGADYGQKNIRCNAVCPGYIETPLLESLTKEMKEALISKHPMGRLGKPEEVAELVLFLSSEKSSFMTGGYYLVDGGYTAV

SEQ ID NO: 11 - DNA, 747 bp, Artificial Sequence
  Engineered sequence
atgggtatcctggacaacaaagtcgcactggttacgggcgctggttcgggcatcggtctggcggtggcacactcctacgctaaagaaggcgctaaagtcattgtgtcagatatcaacgaagacaagggtaataaaaccgttgaagatattaaagcacagggcggtgaagctagttttgtgaaagcggacaccagcaacccggaagaagtggaagccctggttaaacgtacggtcgaaatttatggtcgcctggatgtggcatgcaacaatgctggcattgcgggtgaaaaggcactggctggtgattacggcctggacagctggcgtaaagttctgtctgtgaatctggacggtgtcttctatggctgtaaatacgaactggaacaaatggagaaaaacggcggtggcgttatcgtcaatatggccagcatttgcggtatcgttgcgcagccgctgaactctgcatatacctctgcgaaacacgccgtggttggcctgacgaaaaacattggtgctgattatggccagaaaaacatccgttgcaatgcggtgtgcccgggcgcgattgaaaccccgctgctggaatcactgacgaaagaaatgaaagaagccctgatctcgaaacacccgatgggtcgcctgggcaaaccggaagaagtggcagaactggttctgtttctgagttccgaaaaatcatcgttcatgaccggtggctattacctggtcgatggtggctacacggcagtg

SEQ ID NO: 12 - protein, 249 aa, Artificial Sequence
  Engineered sequence
MGILDNKVALVTGAGSGIGLAVAHSYAKEGAKVIVSDINEDKGNKTVEDIKAQGGEASFVKADTSNPEEVEALVKRTVEIYGRLDVACNNAGIAGEKALAGDYGLDSWRKVLSVNLDGVFYGCKYELEQMEKNGGGVIVNMASICGIVAQPLNSAYTSAKHAVVGLTKNIGADYGQKNIRCNAVCPGAIETPLLESLTKEMKEALISKHPMGRLGKPEEVAELVLFLSSEKSSFMTGGYYLVDGGYTAV

SEQ ID NO: 13 - DNA, 747 bp, Artificial Sequence
  Engineered sequence
atgggtatcctggacaacaaagtcgcactggttacgggcgctggttcgggcatcggtctggcggtggcacactcctacgctaaagaaggcgctaaagtcattgtgtcagatatcaacgaagacaagggtaataaaaccgttgaagatattaaagcacagggcggtgaagctagttttgtgaaagcggacaccagcaacccggaagaagtggaagccctggttaaacgtacggtcgaaatttatggtcgcctggatgtggcatgcaacaatgctggcattgcgggtgaaaaggcactggctggtgattacggcctggacagctggcgtaaagttctgtctgtgaatctggacggtgtcttctatggctgtaaatacgaactggaacaaatggagaaaaacggcggtggcgttatcgtcaatatggccagcatttgcggtatcgttgcgcagccgctgaactctgcatatacctctgcgaaacacgccgtggttggcctgacgaaaaacattggtgctgattatggccagaaaaacatccgttgcaatgcggtgtgcccgggcggcattgaaaccccgctgctggaatcactgacgaaagaaatgaaagaagccctgatctcgaaacacccgatgggtcgcctgggcaaaccggaagaagtggcagaactggttctgtttctgagttccgaaaaatcatcgttcatgaccggtggctattacctggtcgatggtggctacacggcagtg

SEQ ID NO: 14 - protein, 249 aa, Artificial Sequence
  Engineered sequence
MGILDNKVALVTGAGSGIGLAVAHSYAKEGAKVIVSDINEDKGNKTVEDIKAQGGEASFVKADTSNPEEVEALVKRTVEIYGRLDVACNNAGIAGEKALAGDYGLDSWRKVLSVNLDGVFYGCKYELEQMEKNGGGVIVNMASICGIVAQPLNSAYTSAKHAVVGLTKNIGADYGQKNIRCNAVCPGGIETPLLESLTKEMKEALISKHPMGRLGKPEEVAELVLFLSSEKSSFMTGGYYLVDGGYTAV

SEQ ID NO: 15 - DNA, 747 bp, Artificial Sequence
  Engineered sequence
atgggtatcctggacaacaaagtcgcactggttacgggcgctggttcgggcatcggtctggcggtggcacactcctacgctaaagaaggcgctaaagtcattgtgtcagatatcaacgaagacaagggtaataaaaccgttgaagatattaaagcacagggcggtgaagctagttttgtgaaagcggacaccagcaacccggaagaagtggaagccctggttaaacgtacggtcgaaatttatggtcgcctggatgtggcatgcaacaatgctggcattgcgggtgaaaaggcactggctggtgattacggcctggacagctggcgtaaagttctgtctgtgaatctggacggtgtcttctatggctgtaaatacgaactggaacaaatggagaaaaacggcggtggcgttatcgtcaatatggccagcggctgcggtatcgttgcgcagccgctgaactctgcatatacctctgcgaaacacgccgtggttggcctgacgaaaaacattggtgctgattatggccagaaaaacatccgttgcaatgcggtgtgcccgggctacattgaaaccccgctgctggaatcactgacgaaagaaatgaaagaagccctgatctcgaaacacccgatgggtcgcctgggcaaaccggaagaagtggcagaactggttctgtttctgagttccgaaaaatcatcgttcatgaccggtggctattacctggtcgatggtggctacacggcagtg

SEQ ID NO: 16 - protein, 249 aa, Artificial Sequence
  Engineered sequence
MGILDNKVALVTGAGSGIGLAVAHSYAKEGAKVIVSDINEDKGNKTVEDIKAQGGEASFVKADTSNPEEVEALVKRTVEIYGRLDVACNNAGIAGEKALAGDYGLDSWRKVLSVNLDGVFYGCKYELEQMEKNGGGVIVNMASGCGIVAQPLNSAYTSAKHAVVGLTKNIGADYGQKNIRCNAVCPGYIETPLLESLTKEMKEALISKHPMGRLGKPEEVAELVLFLSSEKSSFMTGGYYLVDGGYTAV

SEQ ID NO: 17 - DNA, 747 bp, Artificial Sequence
  Engineered sequence
atgggtatcctggacaacaaagtcgcactggttacgggcgctggttcgggcatcggtctggcggtggcacactcctacgctaaagaaggcgctaaagtcattgtgtcagatatcaacgaagacaagggtaataaaaccgttgaagatattaaagcacagggcggtgaagctagttttgtgaaagcggacaccagcaacccggaagaagtggaagccctggttaaacgtacggtcgaaatttatggtcgcctggatgtggcatgcaacaatgctggcattgcgggtgaaaaggcactggctggtgattacggcctggacagctggcgtaaagttctgtctgtgaatctggacggtgtcttctatggctgtaaatacgaactggaacaaatggagaaaaacggcggtggcgttatcgtcaatatggccagcggctgcggtatcgttgcgcagccgctgaactctgcatatacctctgcgaaacacgccgtggttggcctgacgaaaaacattggtgctgattatggccagaaaaacatccgttgcaatgcggtgtgcccgggcgcgattgaaaccccgctgctggaatcactgacgaaagaaatgaaagaagccctgatctcgaaacacccgatgggtcgcctgggcaaaccggaagaagtggcagaactggttctgtttctgagttccgaaaaatcatcgttcatgaccggtggctattacctggtcgatggtggctacacggcagtg

SEQ ID NO: 18 - protein, 249 aa, Artificial Sequence
  Engineered sequence
MGILDNKVALVTGAGSGIGLAVAHSYAKEGAKVIVSDINEDKGNKTVEDIKAQGGEASFVKADTSNPEEVEALVKRTVEIYGRLDVACNNAGIAGEKALAGDYGLDSWRKVLSVNLDGVFYGCKYELEQMEKNGGGVIVNMASGCGIVAQPLNSAYTSAKHAVVGLTKNIGADYGQKNIRCNAVCPGAIETPLLESLTKEMKEALISKHPMGRLGKPEEVAELVLFLSSEKSSFMTGGYYLVDGGYTAV

SEQ ID NO: 19 - DNA, 747 bp, Artificial Sequence
  Engineered sequence
atgggtatcctggacaacaaagtcgcactggttacgggcgctggttcgggcatcggtctggcggtggcacactcctacgctaaagaaggcgctaaagtcattgtgtcagatatcaacgaagacaagggtaataaaaccgttgaagatattaaagcacagggcggtgaagctagttttgtgaaagcggacaccagcaacccggaagaagtggaagccctggttaaacgtacggtcgaaatttatggtcgcctggatgtggcatgcaacaatgctggcattgcgggtgaaaaggcactggctggtgattacggcctggacagctggcgtaaagttctgtctgtgaatctggacggtgtcttctatggctgtaaatacgaactggaacaaatggagaaaaacggcggtggcgttatcgtcaatatggccagcggctgcggtatcgttgcgcagccgctgaactctgcatatacctctgcgaaacacgccgtggttggcctgacgaaaaacattggtgctgattatggccagaaaaacatccgttgcaatgcggtgtgcccgggcggcattgaaaccccgctgctggaatcactgacgaaagaaatgaaagaagccctgatctcgaaacacccgatgggtcgcctgggcaaaccggaagaagtggcagaactggttctgtttctgagttccgaaaaatcatcgttcatgaccggtggctattacctggtcgatggtggctacacggcagtg

SEQ ID NO: 20 - protein, 249 aa, Artificial Sequence
  Engineered sequence
MGILDNKVALVTGAGSGIGLAVAHSYAKEGAKVIVSDINEDKGNKTVEDIKAQGGEASFVKADTSNPEEVEALVKRTVEIYGRLDVACNNAGIAGEKALAGDYGLDSWRKVLSVNLDGVFYGCKYELEQMEKNGGGVIVNMASGCGIVAQPLNSAYTSAKHAVVGLTKNIGADYGQKNIRCNAVCPGGIETPLLESLTKEMKEALISKHPMGRLGKPEEVAELVLFLSSEKSSFMTGGYYLVDGGYTAV

SEQ ID NO: 21 - DNA, 747 bp, Artificial Sequence
  Engineered sequence
atgggtatcctggacaacaaagtcgcactggttacgggcgctggttcgggcatcggtctggcggtggcacactcctacgctaaagaaggcgctaaagtcattgtgtcagatatcaacgaagacaagggtaataaaaccgttgaagatattaaagcacagggcggtgaagctagttttgtgaaagcggacaccagcaacccggaagaagtggaagccctggttaaacgtacggtcgaaatttatggtcgcctggatgtggcatgcaacaatgctggcattgcgggtgaaaaggcactggctggtgattacggcctggacagctggcgtaaagttctgtctgtgaatctggacggtgtcttctatggctgtaaatacgaactggaacaaatggagaaaaacggcggtggcgttatcgtcaatatggccagcggccatggtatcgttgcgcagccgctgaactctgcatatacctctgcgaaacacgccgtggttggcctgacgaaaaacattggtgctgattatggccagaaaaacatccgttgcaatgcggtgtgcccgggcgcgattgaaaccccgctgctggaatcactgacgaaagaaatgaaagaagccctgatctcgaaacacccgatgggtcgcctgggcaaaccggaagaagtggcagaactggttctgtttctgagttccgaaaaatcatcgttcatgaccggtggctattacctggtcgatggtggctacacggcagtg

SEQ ID NO: 22 - protein, 249 aa, Artificial Sequence
  Engineered sequence
MGILDNKVALVTGAGSGIGLAVAHSYAKEGAKVIVSDINEDKGNKTVEDIKAQGGEASFVKADTSNPEEVEALVKRTVEIYGRLDVACNNAGIAGEKALAGDYGLDSWRKVLSVNLDGVFYGCKYELEQMEKNGGGVIVNMASGHGIVAQPLNSAYTSAKHAVVGLTKNIGADYGQKNIRCNAVCPGAIETPLLESLTKEMKEALISKHPMGRLGKPEEVAELVLFLSSEKSSFMTGGYYLVDGGYTAV